(19) United States
(12) Patent Application Publication
(10) Pub. No.: US 2019/0090756 A1
(43) Pub. Date: Mar. 28, 2019

(54) TECHNOLOGIES FOR SENSING A HEART RATE OF A USER

(71) Applicant: Intel Corporation, Santa Clara, CA (US)

(72) Inventors: Hong Lu, Santa Clara, CA (US); Lama Nachman, Santa Clara, CA (US)

(21) Appl. No.: 15/717,132

(22) Filed: Sep. 27, 2017

(51) Int. Cl. A61B 5/0205; A61B 5/00

(52) U.S. Cl. CPC A61B 5/0205; A61B 5/02438; A61B 5/7275; A61B 5/7221

(57) ABSTRACT

Technologies for determining a heart rate of a user includes a wearable compute device having a heart rate sensor, a motion sensor, a heart rate determination manager, and a heart rate estimator. The wearable compute device generates sensor data indicative of a heart rate of the user and motion data indicative of a motion presently performed by the user. The wearable compute device determines whether to estimate the heart rate of the user based on the heart rate sensor data. The wearable compute device generates an estimated heart rate of the user using a heart rate estimation model and the motion data as an input to the heart rate estimation model in response to a determination to estimate the heart rate.

### BACKGROUND

Wearable compute devices are increasingly equipped with heart rate sensors to monitor the heart rate of the users. Such wearable compute devices may come in many different form factors such as a smart bracelet, smart watch, a wearable monitor, or the like. Heart rate monitoring is often used for sports and well-being applications. In many cases, applications rely on long term heart rate recordings to gauge a user's fitness level, discover habits/trends, estimate caloric expenditure, improve exercise efficiency/performance, or detect abnormalities for early intervention.

Under certain circumstances, the collection of a user's heart rate data may be interrupted or unreliable for various technical reasons and/or human factors. For example, the user may forget to charge the wearable compute device properly or may place sensors of the device in improper or non-optimal locations. Additionally, some operating conditions may pose challenges to the accurate sensing of a user's heart rate (e.g., during excessive user motion, unfavorable lighting conditions, etc.). In such circumstances, the reported heart rate of the user may be erroneous and/or the heart rate functionality of the wearable compute device may be temporarily jeopardized.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a wearable compute device for determining a heart rate of a user;

FIG. 2 is a simplified block diagram of at least one embodiment of an environment that may be established by the wearable compute device of FIG. 1;

FIG. 3 is a simplified flow diagram of at least one embodiment of a method for initializing a heart rate estimation model that may be executed by the wearable compute device of FIGS. 1 and 2; and

FIGS. 4 and 5 are a simplified block diagram of at least one embodiment of a method for determining a heart rate of a user that may be executed by the wearable compute device of FIGS. 1 and 2.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

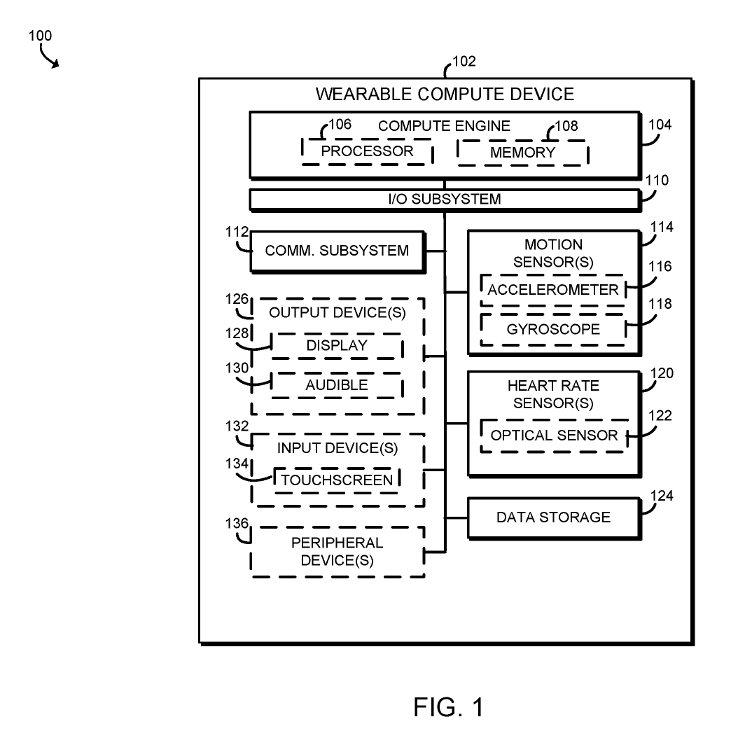

Referring now to FIG. 1, in an illustrative embodiment, a system 100 for heart rate sensing includes a wearable compute device 102 configured to monitor the heart rate of a user while the user is wearing the wearable compute device 102. To do so, the wearable compute device 102 is configured to collect sensor data indicative of a heart rate of the user from one or more heart rate sensors and determine the user's heart rate based on the collected sensor data. However, under certain circumstances (e.g., when the heart rate sensor data is of poor quality), the wearable compute device 102 may estimate the user's heart rate. To do so, the wearable compute device 102 collects motion data indicative of characteristics of the motion of the user (e.g., pace of user's motion, intensity of user's motion, step frequency, type of motion, identified user activity, etc.) and uses the collected motion data as an input to a heart rate estimation model. For example, the wearable compute device 102 may determine whether to estimate the heart rate based on various criteria including, but not limited to, the quality of the sensor data produced by the heart rate sensor, the level of confidence in the heart rate sensor data, the present duty cycle of the heart rate sensor, and/or other parameters to ensure an accurate heart rate reading. In some circumstances, for example, it may be more energy efficient for the wearable compute device 102 to estimate the heart rate of the user using the motion sensor(s) rather than to generate (or estimate) the user's heart rate using (or only using) the heart rate sensor(s) of the device 102. Of course, the wearable compute device 102 may also use a combination of the motion sensor(s) and the heart rate sensor(s) to determine, refine, or otherwise increase the accuracy of the reported heart rate as discussed in more detail below.

As discussed in more detail below, the heart rate estimation model is a mathematical model that generates or produces an estimated heart rate of the user based on motion data produced by one or more motion sensors of the wearable compute device 102. In some embodiments, the heart rate estimation model may be configured to receive the motion data and other data, such as user biometric characteristics, as input. As discussed in more detail below, the heart rate estimation model may be a user-independent model or a user-specific model. For example, in some embodiments, a generalized or user-independent model may be initially used, which is customized to the user over time using machine learning techniques to increase the accuracy of the model. Additionally, in some embodiments, the wearable compute device 102 may communicate with other wearable compute devices 102 or a server (not shown) to exchange data regarding the heart rate estimation model and/or the sensor data and motion data to provide a robust database of sensor data, motion data, and/or heart rate estimation models. It should be appreciated that users have varying body types and characteristics that may cause variances in the data collected by the local sensors of the wearable compute device 102. Many factors may affect a user's average heart rate including biometric characteristics, such as gender, age, height, weight, and/or other factors. Accordingly, the aggregation of data from a plurality of wearable compute devices 102 carried or worn by different users may help to generate or refine a more accurate heart rate estimation model for a particular user. For example, the wearable compute device 102 may access heart rate estimation models of other user's with similar biometric characteristics to accurately provide an initial heart rate estimation model for the particular user as discussed in more detail below.

The wearable compute device 102 may be embodied as any type of compute device capable of being worn or carried by a user and performing the functions described herein. For example, the wearable compute device 102 may be embodied as a smart bracelet, a smartwatch, a fitness tracker, smart glasses, smart clothing or accessories, and/or other computing/communication device capable of being worn by the user. In other embodiments, the wearable compute device 102 may be embodied as a smart phone, a smartphone, a tablet computer, a laptop computer, a notebook, a netbook, an Ultrabook™, a smart device, a personal digital assistant, a mobile Internet device, and/or any other computing/communication device capable of being carried by the user. Depending on the form factor, the wearable compute device 102 may include a strap and/or other coupling mechanism to allow attachment to the user.

As shown in FIG. 1, the illustrative wearable compute device 102 includes a compute engine 104, an input/output (“I/O”) subsystem 110, a communication subsystem 112, one or more motion sensor(s) 114, one or more heart rate sensor(s) 120, and a data storage 124. In some embodiments, the wearable compute device 102 may also include one or more output devices 126, one or more input devices 132, and peripheral devices 136. Of course, the wearable compute device 102 may include other or additional components, such as those commonly found in a typical compute device, in other embodiments. Additionally, in some embodiments, one or more of the illustrative components may be incorporated in, or otherwise form a portion of, another component.

The compute engine 104 may be embodied as any controller, functional block, digital logic, or other component, device, circuitry, or collection thereof capable of performing the functions described herein. In some embodiments, the compute engine 104 may include a processor 106 and a memory 108. In such embodiments, the processor 106 may be embodied as any type of processor capable of performing the functions described herein. For example, the processor 106 may be embodied as a single or multi-core processor(s), digital signal processor, microcontroller, or other processor or processing/controlling circuit. Similarly, the memory 108 may be embodied as any type of volatile or non-volatile memory or data storage capable of performing the functions described herein. In operation, the memory 108 may store various data and software used during operation of the wearable compute device 102 such as operating systems, applications, programs, libraries, and drivers. The memory 108 is communicatively coupled to the processor 106 via the I/O subsystem 110, which may be embodied as circuitry and/or components to facilitate input/output operations with the processor 106, the memory 108, and other components of the wearable compute device 102. For example, the I/O subsystem 110 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 110 may form a portion of a system-on-a-chip (SoC) and be incorporated, along with the processor 106, the memory 108, and other components of the wearable compute device 102, on a single integrated circuit chip. In some embodiments, the memory 108, or portions of the memory 108 may be incorporated into the processor 106.

The communication subsystem 112 may be embodied as any type of communication circuit, device, or collection thereof, capable of enabling communications between the wearable compute device 102 and other remote devices such as other wearable compute devices 102. To do so, the communication subsystem 112 may be configured to use any one or more communication technologies (e.g., wireless or wired communications) and associated protocols (e.g., Ethernet, Bluetooth®, Wi-Fi®, WiMAX, LTE, 5G, etc.) to effect such communication.

The motion sensor(s) 114 may be embodied as any one or more sensors capable of generating or producing motion data indicative of any type of characteristic of the motion of the user while the user performs a present activity. For example, in some embodiments, the motion data produced by the motion sensor(s) 114 may be indicative of a type, an intensity, and/or a pace of the present activity performed by the user. Such monitored activities may include, for example, walking, running, sitting, resting, etc. For example, in some embodiments, the motion sensor(s) 114 may include one or more accelerometers 116 and/or one or more gyroscopes 118. In other embodiments, additional or other sensors may be included in the wearable compute device 102 to generate motion data indicative a characteristic of the motion (e.g., motion intensity) and/or activity presently performed by the user.

The heart rate sensor(s) 120 may be embodied as any one or more sensors capable of generating or producing sensor data indicative of a heart rate of the user of the wearable compute device. For example, in some embodiments, the heart rate sensor(s) 120 may include one or more optical sensors 122 configured to generate heart rate sensor data based on optical signals transmitted through the skin of the user. In other embodiments, additional or other sensors may be included in the wearable compute device 102 to generate sensor data indicative of the heart rate of the user.

The data storage device 124 may be embodied as any type of device or devices configured for short-term or long-term storage of data such as, for example, memory devices and circuits, memory cards, hard disk drives, solid-state drives, non-volatile flash memory, or other data storage devices.

In some embodiments, the wearable compute device 102 may include one or more output devices 126. The output devices 126 may include, or be embodied as, any type of output device capable of providing information to the user of the wearable compute device 102. In the some embodiments, the output devices 126 include a display 128 and/or an audible device 130, but may include additional or other components in other embodiments. The display 128 may be embodied as any type of display capable of displaying information to the user of the wearable compute device 102. For example, the display 128 may be embodied as a liquid crystal display (LCD), a light emitting diode (LED) display, an organic light emitting diode (OLED), a cathode ray tube (CRT) display, a plasma display, and/or other display device. The audible 130 may be embodied as any type of audio generation device, such as a speaker or annunciator, capable of producing sound.

In some embodiments, the wearable compute device 102 may include one or more input devices 132. The input devices 132 may be embodied as any one or more devices capable of receiving an interaction from a user of the wearable compute device 102 and providing an input to the wearable compute device 102 based on such interaction. In some embodiments, the input devices 132 include a touchscreen 134, which may receive input from the user based on a tactile interaction. Additionally, in some embodiments, the touchscreen 134 may form a portion of the display 128. Of course, the input devices 132 may include additional or other types of input devices such as a physical or virtual keyboard, buttons, switches, a mouse, and so forth.

In some embodiments, the wearable compute device 102 may also include one or more peripheral devices 136. The peripheral devices 136 may include any number of additional peripheral or interface devices, such as other input/output devices, storage devices, and so forth. The particular devices included in the peripheral devices 136 may depend on, for example, the type and/or intended use of the wearable compute device 102.

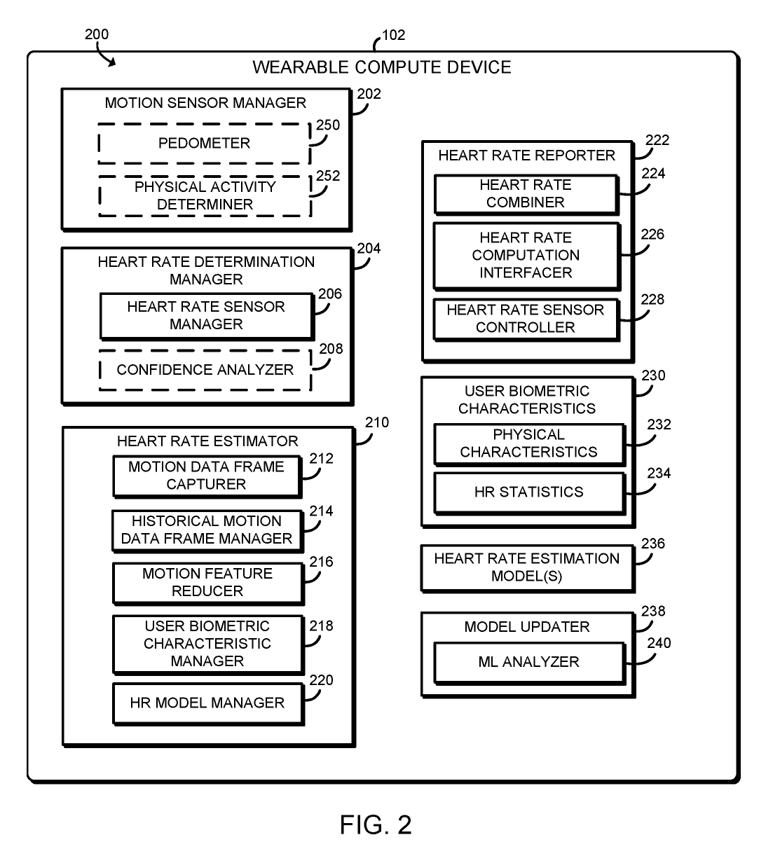

Referring now to FIG. 2, in the illustrative embodiment, the wearable compute device 102 may establish an environment 200 during operation. The illustrative environment 200 includes a motion sensor manager 202, a heart rate determination manager 204, a heart rate estimator 210, a heart rate reporter 222, user biometric characteristics 230, a heart rate estimation model 236, and a model updater 238. Each of the components, logic, and other modules of the environment 200 may be embodied as hardware, firmware, software, or a combination thereof. As such, in some embodiments, one or more of the components of the environment 200 may be embodied as circuitry or collection of electrical devices (e.g., motion sensor manager circuitry 202, heart rate determination circuitry 204, etc.). It should be appreciated that, in some embodiments, one or more of the motion sensor manager 202, the heart rate determination manager 204, the heart rate estimator 210, the heart rate reporter 222, the user biometric characteristics 230, the heart rate estimation model 236, and/or the model updater 238 may form a portion of one or more of the compute engine 104, processor 106, memory 108, communication subsystem 112, motion sensor(s) 114, heart rate sensor(s) 120, and/or other components of the wearable compute device 102. Additionally, in some embodiments, one or more of the illustrative components may form a portion of another component and/or one or more of the illustrative components may be independent of one another. Further, in some embodiments, one or more of the components of the environment 200 may be embodied as virtualized hardware components or emulated architecture, which may be established and maintained by the processor 106 or other components of the wearable compute device 102.

The motion sensor manager 202, which may be embodied as hardware, firmware, software, virtualized hardware, emulated architecture, and/or a combination thereof as discussed above, is configured to receive and manage the motion data produced by the various motion sensor(s) 114. Under certain circumstances, the motion sensor manager 202 may aggregate, preprocess, or otherwise prepare the motion data to be inputted into a heart rate estimation model 236 as described in more detail below. In some embodiments, the motion sensor manager 202 may include various sub-modules configured to determine various aspects of the user's motion based on the motion data. For example, as shown in FIG. 2, the motion sensor manager 202 may include a pedometer 250 and/or a physical activity determiner 252. In such embodiments, the pedometer 250 may be configured to determine step counts or similar data from the motion data, and the physical activity determiner 252 may be configured to determine a type of physical activity (e.g., running, walking, sitting) presently being performed by the user based on the motion data. As discussed above, the motion data may be indicative of any type of characteristic of the motion of the user including, but not limited to, the type, intensity, and pace of the user's present motion. The type of motion may define, for example, the user's present activity (e.g., whether the user is running, walking, etc.). The intensity of the user's motion may define, for example, the exertion exhibited by the user, such as the size of weights a user may lift with a period of time (e.g. the higher the weight, the higher the intensity) or how much a user pushes off the ground while running. The pace of the user's motion may define, for example, how fast a user is performing repetitions for an activity (e.g. how fast a user is performing a bench press) or how frequently a user lands on the ground as the user is walking, running, etc.

The heart rate determination manager 204, which may be embodied as hardware, firmware, software, virtualized hardware, emulated architecture, and/or a combination thereof that is configured to generate or estimate a present heart rate of the user based on sensor data produced by the heart rate sensor 120 and, under some circumstances, and estimated heart rate produced by the heart rate estimator 210 as discussed below. In the illustrative embodiment, the heart rate determination manager 204 includes a heart rate sensor manager 206, and may also include a confidence analyzer 208. The heart rate sensor manager 206 is configured to receive and manage the sensor data produced by the heart rate sensor(s) 120. For example, the heart rate sensor manager 206 may perform various pre-processing techniques on the sensor data produced by the heart rate sensor(s) 120.

Under normal operation conditions, the heart rate determination manager 204 may determine (or estimate) the heart rate of the user based solely on the sensor data produced by the heart rate sensor(s) 120. However, under certain conditions, the heart rate determination manager 204 may determine that the user's heart rate should be estimated based on the sensor data produced by the motion sensor(s) 114. For example, the heart rate determination manager 204 may determine that the user's heart rate should be estimated in response to a determination that the heart rate sensor 120 is malfunctioning, turned-off, damaged, or otherwise producing erroneous sensor data. Additionally or alternatively, the heart rate determination manager 204 may determine whether to estimate the user's heart rate based on the quality of the sensor data produced by the heart rate sensor(s) 120. That is, the heart rate determination manager 204 may determine that that the user's heart rate should be estimated if the quality of the heart rate sensor data is poor (e.g., does not satisfy a reference relationship with one or more threshold quality values). To do so, the heart rate determination manager 204 may analyze any quality of the sensor data produced by the heart rate sensor(s) 120. For example, the heart rate determination manager 204 may compare a signal-to-noise ratio, a peak amplitude, a signal distortion value, and/or other characteristic of the heart rate sensor data to threshold values to determine whether to estimate the user's heart rate. Additionally, in some embodiments, the present sensor data may be compared to historic sensor data (e.g., comparing the presently determined user's heart rate to the user's average heart rate) to determine whether the present sensor data is of a sufficient quality and/or not erroneous. The quality threshold values to which the sensor data is compared may be predetermined, user-selected, or determined/adjusted over time. For example, in some embodiments, the user may set the quality threshold values. In other embodiments, the quality threshold values may be set or adjusted over time based on various criteria including, for example, sensor data aggregated from other wearable compute devices 102.

In some embodiments, the heart rate determination manager 204 may determine to estimate the user's heart rate to improve the efficiency (e.g., improve power consumption) of the wearable compute device 102. For example, the heart rate determination manager 204 may monitor the duty cycle of the heart rate sensor(s) 120 and determine to estimate the user's heart rate if the duty cycle is greater than a reference duty cycle threshold value. That is, if the duty cycle of the heart rate sensor(s) 120 is high, the heart rate determination manager 204 may decrease the duty cycle of the heart rate sensor(s) 120, and reduce overall power consumption, by estimating the heart rate of the user using the motion data produced by the motion sensor(s) 114. The duty cycle threshold value may be predetermined, user-selected, or determined/adjusted over time. For example, in some embodiments, the user may set the duty cycle threshold value depending on the amount of desired power saving. In other embodiments, the duty cycle threshold value may be set or adjusted over time based on various criteria including, for example, data aggregated from other wearable compute devices 102.

In some embodiments, the heart rate determination manager 204 may include a heart rate sensor manager 206 configured to analyze a confidence score, which may be associated with the sensor data produced by the heart rate sensor(s) 120 in some embodiments. The confidence score defines the level of confidence in accuracy of the sensor data produced by the heart rate sensor(s) 120 at that particular point in time. For example, the confidence score of the sensor data produced by the heart rate sensor(s) 120 may be high when the heart rate from the heart rate sensor(s) 120 is similar to the average received heart rate from the heart rate sensor(s) 120 for a given activity performed by the user. In another example, the confidence score of the sensor data produced by the heart rate sensor(s) 120 may be low when the user is moving frequently (e.g. running, other activity that would disturb position of wearable compute device 102 as it is measuring sensor data, etc.). Under such circumstances, the heart rate sensor(s) 120 may not be able to consistently obtain an accurate measurement.

In embodiments in which the heart rate determination manager 204 includes the confidence analyzer 208, the heart rate determination manager 204 may determine to estimate the heart rate of the user based on the confidence score associated with the heart rate sensor data. For example, the heart rate determination manager 204 may determine to estimate the heart rate of the user when the confidence score falls below a confidence score threshold value, which may indicate that the heart rate sensor data is erroneous. The confidence score threshold value may be predetermined, user-selected, or determined/adjusted over time. For example, in some embodiments, the user may set the confidence score threshold value. In other embodiments, the confidence score threshold value may be set or adjusted over time based on various criteria including, for example, data aggregated from other wearable compute devices 102.

The heart rate estimator 210, which may be embodied as hardware, firmware, software, virtualized hardware, emulated architecture, and/or a combination thereof as discussed above, is configured to generate an estimated heart rate of the user based on a request or determination to do so from the heart rate determination manager 204. To do so, as discussed in more detail below, the heart rate estimator 210 estimates the heart rate of the user using a heart rate estimation model 236 with the motion data and, in some embodiments the user biometric characteristics 230, as input to the heart rate estimation model 236. In the illustrative embodiment, the heart rate estimator 210 includes a motion data frame capturer 212, a historical motion data frame manager 214, a motion feature reducer 216, a user biometric characteristic manager 218, and a heart rate (HR) model manager 220.

The motion data frame capturer 212 is configured to capture temporal data frames of the motion sensor data produced by the motion sensor(s) 114. That is, the motion data frame capturer 212 captures the motion sensor data during each window of time. The particular length of the captured temporal data frame may vary depending on the particular implementation, type of motion sensor 114, and/or other criteria. For example, the motion data frame capturer 212 may capture 1 second, 3, seconds, 5 seconds, 10 seconds, etc. of motion data for each temporal data frame. It should be appreciated that the captured motion sensor data may include various features (e.g., peak amplitude, average values, variance between peaks/valleys, etc.) that define the characteristics of the present motion of the user during the captured temporal data frame. In some embodiments, the motion data frame capturer 212 may process the captured temporal data frame to extract the features of the captured motion data and generate a present motion feature vector based on the features of captured motion sensor data.

The historical motion data frame manager 214 is configured to manage, store, and retrieve historical temporal data frames of motion data captured by the motion data frame capturer 212. It should be appreciated that, in addition to the user's present motion (e.g., activity), recent user motion or activity is also a good indication of the user's present heart rate. For example, the user's heart rate may remain elevated or gradually decay after strenuous activity (e.g., running or climbing a flight of stairs). In such a situation, the user's heart rate would be elevated even if the user's current activity is simply standing or sitting. As such, historical motion data is also considered. For example, in the illustrative embodiment, the historical motion data frame manager 214 is configured to store and manage the last few (e.g., the last six) temporally sequential motion data frames relative to the presently captured temporal data frame of the motion data. In such embodiments, the oldest captured temporal data frame may be discarded when a new temporal data frame of the motion data is captured such that the historical motion data frame manager 214 maintains a constant number of historical temporal data frames of the user's motion data. In those embodiments in which the motion data frame capturer 212 generates a motion feature vector for the captured temporal data frame, the historical motion data frame manager 214 may store the generated motion feature vectors as historical motion feature vectors rather than, or in addition to the captured temporal data frames (i.e., the captured motion data).

As discussed in more detail below, the present temporal data frame captured by the motion data frame capturer 212 and the historical temporal data frames managed by the historical motion data frame manager 214 are used as inputs to the heart rate estimation model 236. Due to the use of multiple temporal data frames, the total number of features of the captured data may become quite large. As such, the motion feature reducer 216 is configured to reduce the number of features included in the present and historical temporal data frames (i.e., in the feature vectors corresponding to the temporal data frames). In those embodiments in which the motion data frame capturer 212 is configured generate the motion feature vector for each captured temporal data frame, the motion feature reducer 216 may combine the feature vectors prior to reducing the features contained therein. In embodiments wherein the motion data frame capturer 212 does not generate the motion feature vectors, the motion feature reducer 216 may process and extract the features of the present temporal data frame and the historical data frames prior to reducing the features. The motion feature reducer 216 may utilize any suitable methodology or algorithm to reduce the features of the data frames or feature vectors to generate a finalized, reduced feature vector. For example, in some embodiments, the motion feature reducer 216 may utilize a principal component analysis (PCA) or an auto-encoder algorithm to reduce the features of the feature vectors. It should be appreciated that by reducing the total number of features, the computational load for estimating the user heart rate may likewise be reduced.

The user biometric characteristic manager 218 is configured to manage and retrieve the user biometric characteristics 230 for use with the heart rate estimation model 236 as discussed in more detail below. As shown in FIG. 2, the user biometric characteristics 230 include physical characteristics 232 of the user and heart rate statistics 234 of the user. The physical characteristics 232 may include any type of data indicative of a physical aspect or characteristic of the user that help define an overall fitness profile of the user. For example, the physical characteristics 232 may include, but are not limited to, the user's height, weight, gender, age, medical condition, etc. In some embodiments, the physical characteristics 232 may be supplied by the user and may have varying degrees of granularity. For example, the wearable compute device 102 may prompt the user to supply the physical characteristics 232.

The heart rate statistics 234 may include any type of data indicative of the user's average or median heart rate during various activities (e.g., baseline activities). For example, the heart rate statistics 234 may include the user's median heart rate while stationary, walking, running, etc. In some embodiments, the wearable compute device 102 may prompt the user to supply the heart rate statistics 234. In other embodiments, the wearable compute device 102 may perform an initialization procedure in which the user is requested to perform each of the various activities while the user's average heart rate is determined for the specific, requested activity as discussed in more detail below. In yet other embodiments, average heart rate statistics of individuals matching the user's physical characteristics 232 may be used as an initial average for the particular user.

In some embodiments, the user biometric characteristics 230 may be appended to, incorporated in, or otherwise aggregated with the present and historical data frames. In such embodiments, features of the user biometric characteristics 230 may be extracted and included in the feature vector, which is subsequently reduced by the motion feature reducer 216 to generate the reduced feature vector. That is, in such embodiments, the reduced feature vector may include features of the user's present motion (i.e., the present temporal data frame), the user's historical motion (i.e., the historical temporal data frames), and the user's biometric characteristics 230 (i.e., the user's physical characteristics 232 and/or the user's heart rate statistics 234).

The heart rate model manager 220 is configured to manage the heart rate estimation model(s) 236 used by the wearable compute device 102. The heart rate estimation model(s) 236 may be embodied as any type of mathematical model capable of mapping features of a user's motion to an estimated heart rate. In the illustrative embodiment, the heart rate estimation model 236 is a regression model, but other types of mathematical models may be used in other embodiments. The heart rate model manager 220 is configured to utilize the heart rate estimation model 236 to determine an estimated heart rate of the user. To do so, in the illustrative embodiment, the heart rate model manager 220 uses the reduced feature vector of the user's present and historical motion produced by motion feature reducer 216 as input to the heart rate estimation model 236. As discussed above, the reduced feature vector may also include the user biometric characteristics 230 in some embodiments. Additionally, in some embodiments, the heart rate estimation model 236 may be configured to directly receive the motion data produced by the motion sensors 114 as an input.

The heart rate estimation model 236 may be user-specific or a generic model depending on the particular implementation. Additionally, the wearable compute device 102 may store and maintain different heart rate estimation models 236 for different users. In the illustrative embodiment, the heart rate estimation model 236 is initially embodied as a user-generic model that is customized or personalized to the user over time using a machine learning algorithm as discussed in more detail below. For example, in some embodiments, an initial heart rate estimation model 236 may be selected or otherwise obtained based on the user's biometric characteristics 230 (e.g., on the user's physical characteristics 232). In such embodiments, the wearable compute device 102 may maintain multiple heart rate estimation models 236 to allow selection of one that best matches the particular user (e.g., one that best matches the uses age, height, weight, gender, etc.). Such user-generic heart rate estimation models 236 may be developed offline based on motion data captured from individuals with matching personal characteristics.

The heart rate reporter 222, which may be embodied as hardware, firmware, software, virtualized hardware, emulated architecture, and/or a combination thereof as discussed above, is configured to control the reporting of the estimated heart rate to the user of the wearable compute device 102. For example, the heart rate reporter 222 may display or otherwise produce the estimated heart rate determined by the heart rate estimator 210 to the user on, for example, the display 128. In such embodiments, the display estimated heart rate may be indiscernible from the heart rate determined by the heart rate determination manager 204 based solely on the sensor data produced by the heart rate sensor(s) 120. In other embodiments, the heart rate reporter 222 may notify the user that an estimated heart rate is being displayed or otherwise provided. In addition or alternatively to displaying the estimated heart rate, the heart rate reporter 222 may utilize the estimated heart rate to improve the accuracy of the heart rate determined by the heart rate determination manager 204 and/or otherwise control the functionality of the heart rate sensor (120). To do so, the heart rate reporter 222 illustratively includes a heart rate combiner 224, a heart rate computation interfacer 226, and a heart rate sensor controller 228.

The heart rate combiner 224 may be configured to combine the estimated heart rate produced by the heart rate estimator 210 and the heart rate determined by the heart rate determination manager 204 based on the heart rate sensor data received from the heart rate sensor(s) 120 to produce a final value for the user's heart rate. For example, the heart rate combiner 224 may utilize the estimated heart rate as a verification of the heart rate sensor data received from the heart rate sensor(s) 120. Conversely, the heart rate combiner 224 may utilize the heart rate sensor data received from the heart rate sensor(s) 120 as a verification of the estimated heart rate. In other embodiments, the heart rate combiner 224 may combine the estimated heart rate and the determined heart rate in various ways to generate a final heart rate for the user. For example, the heart rate combiner 224 may average the estimated and determined heart rates to produce a final heart rate value. Of course, the heart rate combiner 224 may utilize more complex or other algorithms to combine the estimated and determined heart rate values to produce the final heart rate value. In some embodiments, the use of the heart rate combiner 224 may be a setting selected by the user in order to provide increased accuracy of measurements on the heart rate.

The heart rate computation interfacer 226 is configured to interface with the heart rate determination manager 204 and supply the estimated heart rate thereto. In such embodiments, the heart rate determination manager 204 may be configured to utilize the estimated heart rate, along with the determined heart rate based on the heart rate sensor data produced by the heart rate sensor(s) 120, to determine a final heart rate value. For example, if a high level of motion is experienced, the heart rate determination manager 204 may utilize the estimated heart rate to disambiguate motion noise and the heart rate signal from the heart rate sensor(s) 120. In such embodiments, the heart rate computation interfacer 226 may be used after a determination that the motion noise exceeds a threshold amount.

The heart rate sensor controller 228 may be configured to control or adjust the heart rate sensor(s) 120 of the wearable compute device 102 based on the estimated heart rate. For example, the heart rate sensor controller 228 may enable the heart rate sensor(s) 120 to use a lower duty cycle by periodically reporting the estimated heart rate in order to reduce overall power consumption. Furthermore, in some embodiments, the heart rate sensor controller 228 may turn the heart rate sensor(s) 120 off during periods of time and report the estimated heart rate during such time.

The model updater 238, which may be embodied as hardware, firmware, software, virtualized hardware, emulated architecture, and/or a combination thereof as discussed above, is configured to update the heart rate estimation model 236 based on various data. In the illustrative embodiment, the model updater 238 includes a machine learning (ML) analyzer 240, which may utilize one or more machine learning algorithms to update the heart rate estimation model 236 based on the sensor data produced by the heart rate sensor(s) 120. For example, the machine learning analyzer 240 may update the heart rate estimation model 236 when high quality sensor data (e.g., heart rate sensor data having a high confidence score) is available. By updating the heart rate estimation model 236, the heart rate estimation model 236 may better estimate the user's heart rate over time and respond to changes in the user's biometrics. For example, over time a user may begin exercising more and such change would be detected by the ML analyzer 240 to update the current heart rate estimation model 236. In some embodiments, the model updater 238 may utilize user-assisted learning techniques wherein the user is requested to provide feedback or confirmation to improve the updating of the heart rate estimation model 236. Additionally, in some embodiments, the model updater 238 may update the user's heart rate characteristics 234, which are used by the heart rate estimation model 236, based on high quality sensor data received from the heart rate sensor(s) 120. For example, the model updater 238 may update the user's median heart rate over time based on heart rate sensor data having a high confidence score.

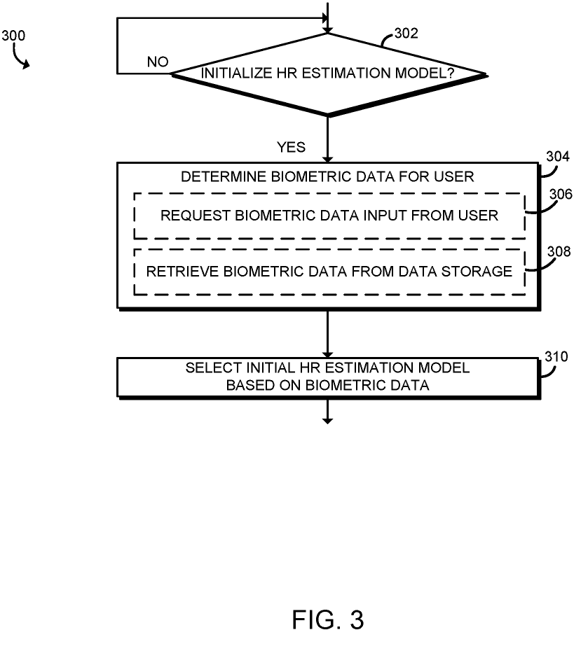

Referring now to FIG. 3, in use, the wearable compute device 102 may execute a method 300 for initializing the heart rate estimation model 236 to be used to estimate the user's heart rate. The method 300 begins with block 302 in which the wearable compute device 102 may determine whether to initialize the heart rate estimator. In some embodiments, the wearable compute device 102 may initialize the heart rate estimation model 236 upon initial power-up or in response to an indication that a new user desires to use the wearable compute device 102 for heart rate monitoring.

If it is determined that a heart rate estimation model 236 is to be initialized, the method 300 advances to block 304. In block 304, the wearable compute device 102 determines the biometric data for the user in order to generate a user profile for the user of the wearable compute device 102. The biometric data may include the physical characteristics 232 and/or the heart rate statistics 234 as described above. In some embodiments, in block 306, the wearable compute device 102 may request biometric data input from the user. The user may, for example, supply the biometric data through the input devices 132 such as a touchscreen 134. In some embodiments, in block 308, the wearable compute device 102 may retrieve biometric data from the data storage 124 (e.g., in the case that the user previously entered biometric data or the wearable compute device 102 gathered the biometric data). Additionally or alternatively, the wearable compute device 102 may retrieve the biometric data from other applications on the wearable compute device 102 that may store the biometric data or a third-party server that contains the biometric data. Furthermore, in some embodiments, the wearable compute device 102 may instruct the user to perform various activities while monitoring the user's heart rate to determine the biometric characteristics (e.g., the heart rate statistics 234 of the user).

In block 310, the wearable compute device 102 selects an initial heart rate estimation model based on the biometric data. As discussed above, the wearable compute device 102 may house multiple user-generic heart rate estimation models 236. In such embodiments, the wearable compute device 102 may select the heart rate estimation model that was produced from individuals having the best matched biometric characteristics as the user. Additionally or alternatively, the wearable compute device 102 may obtain the selected heart rate estimation model from other compute devices, such as a hear rate estimation model server or other wearable compute devices 102. As discussed above, the initial hear rate estimation model is updated overtime using machine learning algorithms to personalize the heart rate estimation model to the specific user.

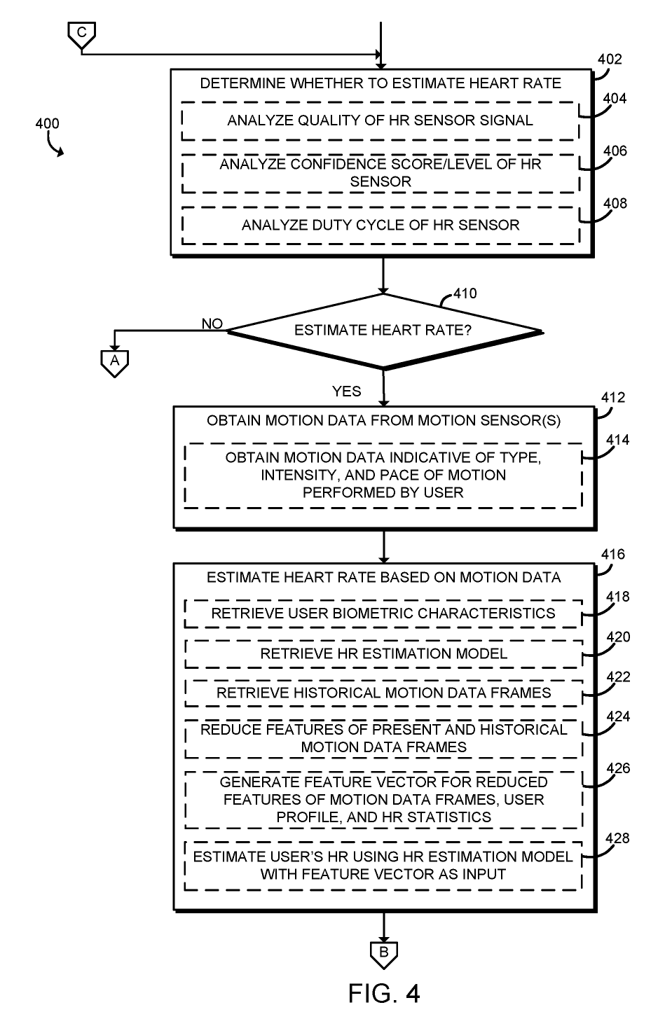

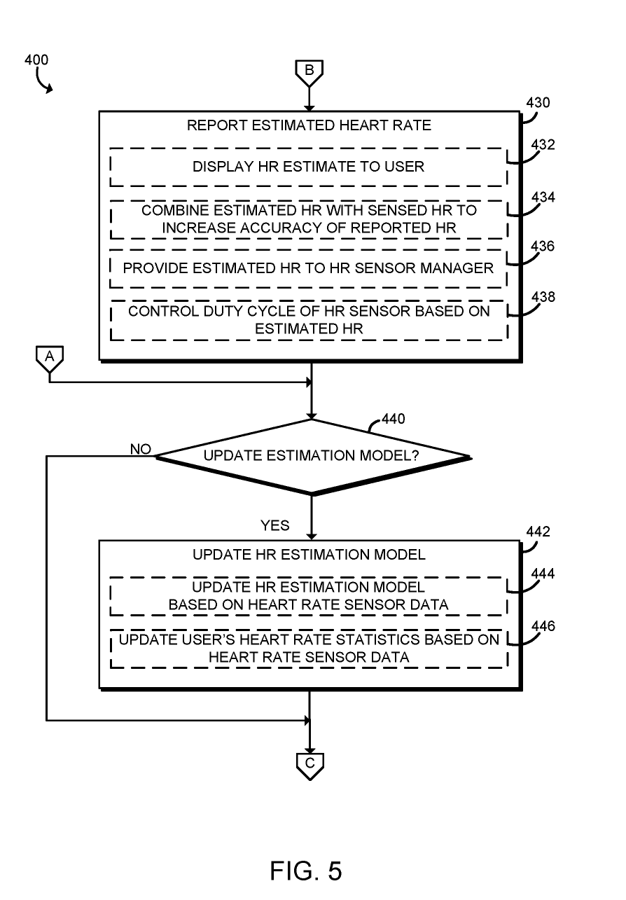

Referring now to FIGS. 4 and 5 in use, the wearable compute device 102 may execute a method 400 for determining a heart rate of a user of the wearable compute device 102. The method 400 begins with block 402 in which the wearable compute device 102 determines whether to estimate the heart rate of the user. To do so, in some embodiments in block 404, the wearable compute device 102 may determine whether to estimate the heart rate by analyzing the quality of the heart rate sensor data/signals received from the heart rate sensor(s) 120. For example, the wearable compute device 102 may analyze the signal-to-noise ratio, peak amplitude, signal distortion, and/or other characteristic of the heart rate sensor data to determine whether to estimate the user's heart rate. As discussed above, the quality of the heart rate sensor signals may be low for various reasons such as when noise is present due to excessive motion of the wearable compute device 102 or poor contact between the heart rate sensor(s) 120 and the skin of the user.

Additionally or alternatively, in some embodiments in block 406, the wearable compute device 102 may determine whether to estimate the user's heart rate by analyzing the confidence score/level of the heart rate sensor data produced by the heart rate sensor(s) 120. As discussed above, the confidence score defines the level of confidence in the accuracy of the sensor data produced by the heart rate sensor(s) 120 at that particular point in time. As such, the wearable compute device 102 may compare the confidence score to a confidence threshold value to determine whether to estimate the user's heart rate. In embodiments wherein the heart rate sensor data does not include a confidence score associated with it, the wearable compute device 102 may infer a confidence score for the present heart rate sensor data by comparing it to a running average heart rate of the user. In such embodiments, if the user's present heart rate value is different from the user's present heart rate average by a threshold amount, the wearable compute device 102 may determine that the heart rate sensor data is erroneous and proceed to estimate the user's heart rate.

Additionally, in some embodiments in block 408, the wearable compute device 102 may determine whether to estimate the heart rate by analyzing the duty cycle of the heart rate sensor. For example, the wearable compute device 102 may determine to estimate the user's heart rate if the duty cycle of the heart rate sensor(s) 120 is greater than a reference threshold value. It should be appreciated that, by reducing the duty cycle of the heart rate sensor(s) 120, the overall power consumption of the wearable compute device 102 may be reduced.

In block 410, the wearable compute device 102 determines whether to proceed with estimating the user's heart rate based on the analysis performed in block 402. If it is determined that estimation of the user's heart rate is required, the method 400 advances to block 412. If it is determined that estimation of the heart rate is not required, the method 400 advance to block 440 of FIG. 5 discussed below.

In block 412, the wearable compute device 102 receives or obtains motion data from the motion sensor(s) 114. As discussed above, the motion data may be embodied as any type of data indicative of one or more characteristic of a motion of the user. For example, in block 414, the motion data may be indicative of a type, an intensity, and/or a pace of the present motion performed by the user depending on, for example, the types of motion sensor(s) 114 included in the wearable compute device 102.

Subsequently in block 416, the wearable compute device 102 estimates the user's heart rate based on the motion data obtained in block 412. To do so, the wearable compute device 102 utilizes the heart rate estimation model 236 with the motion data (e.g., a feature vector based on the motion data) as input to the heart rate estimation model 236 to estimate the user's heart rate. Of course, as discussed above, the heart rate estimation model 236 may use additional data as input in some embodiments. As such, in the illustrative embodiment, the wearable compute device 102 executes blocks 418-428 to estimate the user's heart rate.

In block 418, the wearable compute device 102 retrieves the user biometric characteristic 230. As discussed above, the biometric characteristics 230 may include the physical characteristics 232 and the heart rate characteristics 234 of the user. Additionally, in block 420, the wearable compute device 102 retrieves the heart rate estimation model 236 associated with the user. As discussed above, depending on the length of use of the wearable compute device, the heart rate estimation model 236 used in block 416 to estimate the user's heart rate may be a user-generic model or may be customized to the user due to prior use and/or initialization procedures.

Subsequently, in block 422, the wearable compute device 102 retrieves the historical temporal motion data frames. As discussed above, the historical motion data frames may include the last few (e.g., the last six) temporally sequential motion data frames relative to the presently captured temporal data frame of the motion data. Again, as discussed above, each temporal motion data frame is embodied as a window of time of captured motion data produced by the motion sensor(s) 114. In block 424, the wearable compute device 102 reduces the features of the present and historical temporal motion data frames. As discussed above, the temporal data frames may be stored as raw motion data captured during each corresponding temporal data frames or as feature vectors. If the temporal data frames are stored as raw motion data, the wearable compute device 102 processes the temporal data frames to generate a feature vector including the features of the present temporal motion data frame and the historical motion data frame. Additionally, as discussed above, the feature vector may also include features based on the user biometric characteristics 230 retrieved in block 418. Regardless, in block 424, the wearable compute device 102 reduces the features of the feature vector to generate a final feature vector that includes features of the present and historical temporal motion data frames, the user's physical characteristics, and the user's heart rate statistics. The wearable compute device 102 condenses or reduces the features of the various data to provide a simpler calculation to estimate the heart rate. The reduced, final feature vector is used as an input to the estimation model in block 428 as discussed above.

After the user's estimated heart rate is determined in block 416, the method 400 advances to block 430 of FIG. 5 in which the wearable compute device 102 reports the estimated heart rate. For example, in some embodiments in block 432, the wearable compute device 102 may display the estimated heart rate to the user (e.g., via display 128). Additionally or alternatively, the wearable compute device 102 may combine the estimated heart rate with the heart rate determined based on the heart rate sensor data received from the heart rate sensor(s) 120 to produce a final value for the user's heart rate. For example, the estimated heart rate may be used as a verification of the heart rate determined based on the heart rate sensor data or vice versa. Alternatively, the estimated heart rate may be mathematically combined (e.g., averaged) with the heart rate determined based on the heart rate sensor data received from the heart rate sensor(s) 120 to calculate a final heart rate value.

Additionally or alternatively, in block 436, the estimated heart rate may be used to perform a more accurate calculation of the user's heart rate based on the heart rate sensor data received from the heart rate sensor(s) 120. For example, as discussed above, the estimated heart rate may be used to account for or remove noise from the heart rate sensor data. Further, in some embodiments, the wearable compute device 102 may control the duty cycle of the heart rate sensor(s) 120 based on the user's estimated heart rate in block 438. For example, the duty cycle of the heart rate sensor(s) 120 may be reduced by relying, periodically, on the estimated heart rate. In some embodiments, the heart rate sensors(s) 120 may be completely turned off during periods in which the estimated heart rate has a high confidence.

Subsequently, in block 440, the wearable compute device 102 determines whether to update the heart rate estimation model 236. If not, the method 400 loops back to block 402 to again determine whether the estimate the user's heart rate. However, if the heart rate estimation model 236 is to be updated, the method 400 advances to block 442 in which the heart rate estimation model 236 is updated. To do so, the wearable compute device 102 may employ a machine learning or other training algorithm to update the heart rate estimation model based on heart rate sensor data produced by the heart rate sensors in block 444. That is, as discussed above, when the heart rate sensor data is of high quality (e.g., has a high confidence value associated with it), the measured heart rate sensor data may be used to update the heart rate estimation model 236. Additionally, in block 446, the user's heart rate statistics may be updated using the high quality heart rate sensor data. For example, the user's median heart rate or maximum heart rate may be updated based on heart rate sensor data having a high confidence score. In some embodiments, due to the complexity of the heart rate estimation model 236, the model 236 may be updated offline. For example, the heart rate sensor data may be batched, sent to another higher performance device (e.g., an update server), and the high performance device may perform the updating of the heart rate estimation model 236. In such embodiments, the updated heart rate estimation model 236 may subsequently be downloaded by the wearable compute device 102. Regardless, after the heart rate estimation model 236 has been updated in block 442, the method 400 loops back to block 402 to again determine whether the estimate the user's heart rate.

### Examples

Illustrative examples of the technologies disclosed herein are provided below. An embodiment of the technologies may include any one or more, and any combination of, the examples described below.

Example 1 includes a wearable compute device for determining a heart rate of a user. The wearable compute device includes a heart rate sensor to produce sensor data indicative of a heart rate of the user; a motion sensor to produce motion data indicative of a motion presently performed by the user; a heart rate determination manager to determine whether to estimate the heart rate of the user based on the sensor data produced by the heart rate sensor; and a heart rate estimator to generate an estimated heart rate of the user using a heart rate estimation model and the motion data as input to the heart rate estimation model in response to a determination to estimate the heart rate.

Example 2 includes the subject matter of Example 1, and e of claim 1, to determine whether to estimate the heart rate of the user comprises to analyze a quality of the sensor data produced by the heart rate sensor; and determine to estimate the heart rate of the user in response to a determination that the quality of the sensor data satisfies a reference relationship with a threshold quality value.

Example 3 includes the subject matter of Example 1 or 2, and wherein the threshold quality value is at least one of a signal-to-noise value, a peak amplitude value, or a signal distortion value.

Example 4 includes the subject matter of any of Examples 1-3, and wherein to determine whether to estimate the heart rate of the user comprises to determine to estimate the heart rate of the user in response to a determination that the heart rate sensor is not functioning.

Example 5 includes the subject matter of any of Examples 1-4, and wherein to determine whether to estimate the heart rate of the user comprises to analyze a confidence score associated with the sensor data produced by the heart rate sensor; and determine to estimate the heart rate of the user in response to a determination that the confidence score satisfies a predetermined relationship with a confidence score threshold value.

Example 6 includes the subject matter of any of Examples 1-5, and wherein to determine whether to estimate the heart rate of the user comprises to analyze a duty cycle of the heart rate sensor; and determine to estimate the heart rate of the user in response to a determination that the duty cycle satisfies a predetermined relationship with a duty cycle threshold value.

Example 7 includes the subject matter of any of Examples 1-6, and wherein the motion data is indicative of at least one of a type, an intensity, or a pace of the motion presently performed by the user.

Example 8 includes the subject matter of any of Examples 1-7, and wherein to generate the estimated heart rate comprises to generate the estimated heart rate of the user using the motion data and biometric characteristic data as input to the heart rate estimation model, wherein the biometric characteristic data is indicative of one or more biometric characteristics of the user.

Example 9 includes the subject matter of any of Examples 1-8, and wherein to generate the estimated heart rate comprises to generate the estimated heart rate of the user using the motion data and heart rate statistical data as input to the heart rate estimation model, wherein the heart rate statistical data is indicative of an average heart rate of the user while performing a corresponding activity.

Example 10 includes the subject matter of any of Examples 1-9, and wherein to generate the estimated heart rate comprises to generate the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity.

Example 11 includes the subject matter of any of Examples 1-10, and wherein to generate the estimated heart rate comprises to capture a temporal data frame of the motion data, wherein the temporal data frame includes features indicative of the motion presently performed by the user; retrieve one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured; reduce the number of features included in the temporal data frame and the one or more historical temporal data frames; generate a feature vector based on the reduced number of features; and generate the estimated heart rate of the user using the heart rate estimation model and the feature vector as an input to the heart rate estimation model.

Example 12 includes the subject matter of any of Examples 1-11, and wherein to generate the feature vector comprises to generate a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

Example 13 includes the subject matter of any of Examples 1-12, and further comprising a display; and a heart rate reporter to display the estimated heart rate on the display.

Example 14 includes the subject matter of any of Examples 1-13, and wherein the heart rate determination manager is further to generate a final heart rate value based on the estimated heart rate and the sensor data produced by the heart rate sensor.

Example 15 includes the subject matter of any of Examples 1-14, and wherein the heart rate estimator is further to provide the estimated heart rate to the heart rate determination manager of the wearable compute device, wherein the heart rate determination manager is to generate a heart rate value based on the sensor data produced by the heart rate sensor.

Example 16 includes the subject matter of any of Examples 1-15, and further comprising a heart rate reporter to adjust a duty cycle of the heart rate sensor of a heart rate determination manager of the wearable compute device based on the estimated heart rate, wherein the heart rate determination manager is to generate a heart rate value based on the sensor data produced by the heart rate sensor.

Example 17 includes the subject matter of any of Examples 1-16, and further comprising a model updater to update the heart rate estimation model subsequent to generation of the estimated heart rate.

Example 18 includes the subject matter of any of Examples 1-17, and wherein to update the heart rate estimation model comprises to update the heart rate estimation model based on at least one of the estimated heart rate, the motion data, and the sensor data produced by the heart rate sensor.

Example 19 includes a method for determining a heart rate of a user of a wearable compute device. The method includes determining, by the wearable compute device, whether to estimate the heart rate of the user based on sensor data produced by a heart rate sensor of the wearable compute device; obtaining, by the wearable compute device, motion data produced by one or more motion sensors of the wearable compute device, wherein the motion data is indicative of a motion presently performed by the user; and generating, by the wearable compute device, an estimated heart rate of the user using a heart rate estimation model and the motion data as input to the heart rate estimation model in response to a determination to estimate the heart rate.

Example 20 includes the subject matter of Example 19, and wherein determining whether to estimate the heart rate of the user comprises analyzing, by the wearable compute device, a quality of the sensor data produced by the heart rate sensor; and determining, by the wearable compute device, to estimate the heart rate of the user in response to a determination that the quality of the sensor data satisfies a reference relationship with a threshold quality value.

Example 21 includes the subject matter of Example 19 or 20, and wherein the threshold quality value is a signal-to-noise value, a peak amplitude value, or a signal distortion value.

Example 22 includes the subject matter of any of Examples 19-21, and wherein determining whether to estimate the heart rate of the user comprises determining to estimate the heart rate of the user in response to a determination that the heart rate sensor is not functioning.

Example 23 includes the subject matter of any of Examples 19-22, and wherein determining whether to estimate the heart rate of the user comprises analyzing a confidence score associated with the sensor data produced by the heart rate sensor; and determining to estimate the heart rate of the user in response to a determination that the confidence score satisfies a predetermined relationship with a confidence score threshold value.

Example 24 includes the subject matter of any of Examples 19-23, and wherein determining whether to estimate the heart rate of the user comprises analyzing a duty cycle of the heart rate sensor; and determining to estimate the heart rate of the user in response to a determination that the duty cycle satisfies a predetermined relationship with a duty cycle threshold value.

Example 25 includes the subject matter of any of Examples 19-24, and wherein the motion data is indicative of at least one of a type, an intensity, or a pace of the motion presently performed by the user.

Example 26 includes the subject matter of any of Examples 19-25, and wherein generating the estimated heart rate comprises generating the estimated heart rate of the user using the motion data and biometric characteristic data as input to the heart rate estimation model, wherein the biometric characteristic data is indicative of one or more biometric characteristics of the user.

Example 27 includes the subject matter of any of Examples 19-26, and wherein generating the estimated heart rate comprises generating the estimated heart rate of the user using the motion data and heart rate statistical data as input to the heart rate estimation model, wherein the heart rate statistical data is indicative of an average heart rate of the user while performing a corresponding activity.

Example 28 includes the subject matter of any of Examples 19-27, and wherein generating the estimated heart rate comprises generating the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity

Example 29 includes the subject matter of any of Examples 19-28, and wherein generating the estimated heart rate comprises capturing, by the wearable compute device, a temporal data frame of the motion data, wherein the temporal data frame includes features indicative of the motion presently performed by the user; retrieving, by the wearable compute device, one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured; reducing, by the wearable compute device, the number of features included in the temporal data frame and the one or more historical temporal data frames; generating, by the wearable compute device, a feature vector based on the reduced number of features; and generating, by the wearable compute device, the estimated heart rate of the user using the heart rate estimation model and the feature vector as an input to the heart rate estimation model.

Example 30 includes the subject matter of any of Examples 19-29, and wherein generating the feature vector comprises generating a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

Example 31 includes the subject matter of any of Examples 19-30, and further comprising displaying the estimated heart rate on a display of the wearable compute device.

Example 32 includes the subject matter of any of Examples 19-31, and further comprising generating, by the wearable compute device, a final heart rate value based on the estimated heart rate and the sensor data produced by the heart rate sensor.

Example 33 includes the subject matter of any of Examples 19-32, and further comprising providing the estimated heart rate to the heart rate determination manager of the wearable compute device, wherein the heart rate determination manager is to generate a heart rate value based on the sensor data produced by the heart rate sensor.

Example 34 includes the subject matter of any of Examples 19-33, and further comprising adjusting, by the wearable compute device, a duty cycle of the heart rate sensor or of a heart rate determination manager of the wearable compute device based on the estimated heart rate, wherein the heart rate determination manager is to generate a heart rate value based on the sensor data produced by the heart rate sensor.

Example 35 includes the subject matter of any of Examples 19-34, and further comprising updating, by the wearable compute device, the heart rate estimation model subsequent to generation of the estimated heart rate.

Example 36 includes the subject matter of any of Examples 19-35, and wherein updating the heart rate estimation model comprises updating the heart rate estimation model based on at least one of the estimated heart rate, the motion data, and the sensor data produced by the heart rate sensor.

Example 37 includes one or more machine readable storage media comprising a plurality of instructions stored thereon that in response to being executed result in a compute device performing the method of any of Examples 19-36.

Example 38 includes a wearable compute device for determining a heart rate of a user. The wearable compute device includes means for determining whether to estimate the heart rate of the user based on sensor data produced by a heart rate sensor of the wearable compute device; means for obtaining motion data produced by one or more motion sensors of the wearable compute device, wherein the motion data is indicative of a motion presently performed by the user; and means for generating an estimated heart rate of the user using a heart rate estimation model and the motion data as input to the heart rate estimation model in response to a determination to estimate the heart rate.

Example 39 includes the subject matter of Example 38, and wherein the means for determining whether to estimate the heart rate of the user comprises means for analyzing a quality of the sensor data produced by the heart rate sensor; and means for determining to estimate the heart rate of the user in response to a determination that the quality of the sensor data satisfies a reference relationship with a threshold quality value.

Example 40 includes the subject matter of Example 38 or 39, and wherein the threshold quality value is a signal-to-noise value, a peak amplitude value, or a signal distortion value.

Example 41 includes the subject matter of any of Examples 38-40, and wherein the means for determining whether to estimate the heart rate of the user comprises means for determining to estimate the heart rate of the user in response to a determination that the heart rate sensor is not functioning.

Example 42 includes the subject matter of any of Examples 38-41, and wherein the means for determining whether to estimate the heart rate of the user comprises means for analyzing a confidence score associated with the sensor data produced by the heart rate sensor; and means for determining to estimate the heart rate of the user in response to a determination that the confidence score satisfies a predetermined relationship with a confidence score threshold value.

Example 43 includes the subject matter of any of Examples 38-42, and wherein the means for determining whether to estimate the heart rate of the user comprises means for analyzing a duty cycle of the heart rate sensor; and means for determining to estimate the heart rate of the user in response to a determination that the duty cycle satisfies a predetermined relationship with a duty cycle threshold value.

Example 44 includes the subject matter of any of Examples 38-43, and wherein the motion data is indicative of at least one of a type, an intensity, or a pace of the motion presently performed by the user.

Example 45 includes the subject matter of any of Examples 38-44, and wherein the means for generating the estimated heart rate comprises means for generating the estimated heart rate of the user using the motion data and biometric characteristic data as input to the heart rate estimation model, wherein the biometric characteristic data is indicative of one or more biometric characteristics of the user.

Example 46 includes the subject matter of any of Examples 38-45, and wherein the means for generating the estimated heart rate comprises means for generating the estimated heart rate of the user using the motion data and heart rate statistical data as input to the heart rate estimation model, wherein the heart rate statistical data is indicative of an average heart rate of the user while performing a corresponding activity.

Example 47 includes the subject matter of any of Examples 38-46, and wherein the means for generating the estimated heart rate comprises means for generating the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity

Example 48 includes the subject matter of any of Examples 38-47, and wherein the means for generating the estimated heart rate comprises means for capturing a temporal data frame of the motion data, wherein the temporal data frame includes features indicative of the motion presently performed by the user; means for retrieving one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured; means for reducing the number of features included in the temporal data frame and the one or more historical temporal data frames; means for generating a feature vector based on the reduced number of features; and means for generating the estimated heart rate of the user using the heart rate estimation model and the feature vector as an input to the heart rate estimation model.

Example 49 includes the subject matter of any of Examples 38-48, and wherein the means for generating the feature vector comprises means for generating a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

Example 50 includes the subject matter of any of Examples 38-49, and further comprising means for displaying the estimated heart rate on a display of the wearable compute device.

Example 51 includes the subject matter of any of Examples 38-50, and further comprising means for generating, by the wearable compute device, a final heart rate value based on the estimated heart rate and the sensor data produced by the heart rate sensor.

Example 52 includes the subject matter of any of Examples 38-51, and further comprising means for providing the estimated heart rate to the heart rate determination manager of the wearable compute device, wherein the heart rate determination manager is to generate a heart rate value based on the sensor data produced by the heart rate sensor.

Example 53 includes the subject matter of any of Examples 38-52, and further comprising means for adjusting a duty cycle of the heart rate sensor or of a heart rate determination manager of the wearable compute device based on the estimated heart rate, wherein the heart rate determination manager is to generate a heart rate value based on the sensor data produced by the heart rate sensor.

Example 54 includes the subject matter of any of Examples 38-53, and further comprising means for updating the heart rate estimation model subsequent to generation of the estimated heart rate.

Example 55 includes the subject matter of any of Examples 38-54, and wherein the means for updating the heart rate estimation model comprises means for updating the heart rate estimation model based on at least one of the estimated heart rate, the motion data, and the sensor data produced by the heart rate sensor.

1. A wearable compute device for determining a heart rate of a user, the wearable compute device comprising:
a heart rate sensor to produce sensor data indicative of a heart rate of the user;
a motion sensor to produce motion data indicative of a motion presently performed by the user;
a heart rate determination manager to determine whether to estimate the heart rate of the user based on the sensor data produced by the heart rate sensor; and
a heart rate estimator to generate an estimated heart rate of the user using a heart rate estimation model and the motion data as input to the heart rate estimation model in response to a determination to estimate the heart rate.

2. The wearable compute device of claim 1, wherein to determine whether to estimate the heart rate of the user comprises to:
analyze a quality of the sensor data produced by the heart rate sensor; and
determine to estimate the heart rate of the user in response to a determination that the quality of the sensor data satisfies a reference relationship with a threshold quality value.

3. The wearable compute device of claim 1, wherein to determine whether to estimate the heart rate of the user comprises to:
analyze a confidence score associated with the sensor data produced by the heart rate sensor; and
determine to estimate the heart rate of the user in response to a determination that the confidence score satisfies a predetermined relationship with a confidence score threshold value.

4. The wearable compute device of claim 1, wherein to determine whether to estimate the heart rate of the user comprises to:
analyze a duty cycle of the heart rate sensor; and
determine to estimate the heart rate of the user in response to a determination that the duty cycle satisfies a predetermined relationship with a duty cycle threshold value.

5. The wearable compute device of claim 1, wherein to generate the estimated heart rate comprises to generate the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity.

6. The wearable compute device of claim 1, wherein to generate the estimated heart rate comprises to:
capture a temporal data frame of the motion data, wherein the temporal data frame includes features indicative of the motion presently performed by the user;
retrieve one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured;
reduce the number of features included in the temporal data frame and the one or more historical temporal data frames;
generate a feature vector based on the reduced number of features; and
generate the estimated heart rate of the user using the heart rate estimation model and the feature vector as an input to the heart rate estimation model.

7. The wearable compute device of claim 6, wherein to generate the feature vector comprises to generate a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

8. The wearable compute device of claim 1, further comprising a heart rate reporter to adjust a duty cycle of the heart rate sensor of a heart rate determination manager of the wearable compute device based on the estimated heart rate, wherein the heart rate determination manager is to generate a heart rate value based on the sensor data produced by the heart rate sensor.

9. The wearable compute device of claim 1, further comprising a model updater to update the heart rate estimation model subsequent to generation of the estimated heart rate.

10. A method for determining a heart rate of a user of a wearable compute device, the method comprising:
determining, by the wearable compute device, whether to estimate the heart rate of the user based on sensor data produced by a heart rate sensor of the wearable compute device;
obtaining, by the wearable compute device, motion data produced by one or more motion sensors of the wearable compute device, wherein the motion data is indicative of a motion presently performed by the user; and
generating, by the wearable compute device, an estimated heart rate of the user using a heart rate estimation model and the motion data as input to the heart rate estimation model in response to a determination to estimate the heart rate.

11. The method of claim 10, wherein determining whether to estimate the heart rate of the user comprises:
analyzing, by the wearable compute device, a quality of the sensor data produced by the heart rate sensor; and
determining, by the wearable compute device, to estimate the heart rate of the user in response to a determination that the quality of the sensor data satisfies a reference relationship with a threshold quality value.

12. The method of claim 10, wherein determining whether to estimate the heart rate of the user comprises:
analyzing a confidence score associated with the sensor data produced by the heart rate sensor; and
determining to estimate the heart rate of the user in response to a determination that the confidence score satisfies a predetermined relationship with a confidence score threshold value.

13. The method of claim 10, wherein determining whether to estimate the heart rate of the user comprises:
analyzing a duty cycle of the heart rate sensor; and
determining to estimate the heart rate of the user in response to a determination that the duty cycle satisfies a predetermined relationship with a duty cycle threshold value.

14. The method of claim 10, wherein generating the estimated heart rate comprises generating the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity

15. The method of claim 10, wherein generating the estimated heart rate comprises:
capturing, by the wearable compute device, a temporal data frame of the motion data, wherein the temporal data frame includes features indicative of the motion presently performed by the user;
retrieving, by the wearable compute device, one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured;
reducing, by the wearable compute device, the number of features included in the temporal data frame and the one or more historical temporal data frames;
generating, by the wearable compute device, a feature vector based on the reduced number of features; and
generating, by the wearable compute device, the estimated heart rate of the user using the heart rate estimation model and the feature vector as an input to the heart rate estimation model.

16. The method of claim 15, wherein generating the feature vector comprises generating a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

17. The method of claim 10, further comprising adjusting, by the wearable compute device, a duty cycle of the heart rate sensor or of a heart rate determination manager of the wearable compute device based on the estimated heart rate, wherein the heart rate determination manager is to generate a heart rate value based on the sensor data produced by the heart rate sensor.

18. One or more machine-readable storage media comprising a plurality of instructions stored thereon that, when executed, cause a wearable compute device to:
determine whether to estimate the heart rate of the user based on sensor data produced by a heart rate sensor of the wearable compute device;
obtain motion data produced by one or more motion sensors of the wearable compute device, wherein the motion data is indicative of a motion presently performed by the user; and
generate an estimated heart rate of the user using a heart rate estimation model and the motion data as input to the heart rate estimation model in response to a determination to estimate the heart rate.

19. The one or more machine-readable storage media of claim 18, wherein to determine whether to estimate the heart rate of the user comprises to:
analyze a quality of the sensor data produced by the heart rate sensor; and
determine to estimate the heart rate of the user in response to a determination that the quality of the sensor data satisfies a reference relationship with a threshold quality value.

20. The one or more machine-readable storage media of claim 18, wherein to determine whether to estimate the heart rate of the user comprises to:
analyze a confidence score associated with the sensor data produced by the heart rate sensor; and
determine to estimate the heart rate of the user in response to a determination that the confidence score satisfies a predetermined relationship with a confidence score threshold value.

21. The one or more machine-readable storage media of claim 18, wherein to determine whether to estimate the heart rate of the user comprises to:
analyze a duty cycle of the heart rate sensor; and
determine to estimate the heart rate of the user in response to a determination that the duty cycle satisfies a predetermined relationship with a duty cycle threshold value.

22. The one or more machine-readable storage media of claim 18, wherein to generate the estimated heart rate comprises to generate the estimated heart rate of the user using the motion data, biometric characteristic data, and heart rate statistical data as input to the heart rate estimation model, wherein the biometric characteristic data is indicative of one or more biometric characteristics of the user and the heart rate statistical data is indicative of an average heart rate of the user while performing a corresponding activity

23. The one or more machine-readable storage media of claim 18, wherein to generate the estimated heart rate comprises to:
capture a temporal data frame of the motion data, wherein the temporal data frame includes features indicative of the motion presently performed by the user;
retrieve one or more historical temporal data frames of motion data, wherein the temporal data frame and the one or more historical temporal data frames are temporally sequential and wherein each of the historical temporal data frames includes features indicative of a motion performed by the user when the corresponding historical temporal data frame was captured;
reduce the number of features included in the temporal data frame and the one or more historical temporal data frames;
generate a feature vector based on the reduced number of features; and
generate the estimated heart rate of the user using the heart rate estimation model and the feature vector as an input to the heart rate estimation model.

24. The one or more machine-readable storage media of claim 23, wherein to generate the feature vector comprises to generate a feature vector based on the reduced number of features, biometric characteristic data, and heart rate statistical data, wherein the biometric characteristic data is indicative of one or more biometric characteristics of the user and the heart rate statistical data is indicative of an average heart of the user while performing a corresponding activity.

25. The one or more machine-readable storage media of claim 18, wherein the plurality of instructions, when executed, further cause the wearable compute device to adjust a duty cycle of the heart rate sensor or of a heart rate determination manager of the wearable compute device based on the estimated heart rate, wherein the heart rate determination manager is to generate a heart rate value based on the sensor data produced by the heart rate sensor.
